(19) United States
(12) Patent Application Publication
(10) Pub. No.: US 2024/0319128 A1
(43) Pub. Date: Sep. 26, 2024

(54) WIRELESS SENSOR BASED ON LC RESONATOR FOR MONITORING SOIL WATER CONTENT

(71) Applicant: Auburn University, Auburn, AL (US)

(72) Inventors: Zhongyang CHENG, Auburn, AL (US); Jiachen LIU, Auburn, AL (US); Wei YI, Auburn, AL (US)

(21) Appl. No.: 18/613,807

(22) Filed: Mar. 22, 2024

Related U.S. Application Data
(60) Provisional application No. 63/454,369, filed on Mar. 24, 2023.

(51) Int. Cl. G01N 27/22; G01N 33/24

(52) U.S. Cl. CPC G01N 27/225; G01N 33/246

(57) ABSTRACT

In at least one illustrative embodiment, a system for wireless soil humidity sensing includes one or more wireless sensors. Each sensor includes a capacitor and an inductor, the capacitor having a dielectric material with a high relative dielectric permittivity that changes in response to changes in environmental humidity. The dielectric material may be a ceramic material such as a core-shell composite including barium titanate (BaTiO₃) and silicon dioxide (SiO₂). The capacitor may include two opposing sides, with a pair of electrodes positioned on one of the sides. An encapsulation layer such as epoxy may cover the side of the capacitor having the electrode. Sensors may be distributed throughout the soil in an area such as a farm field. The resonant frequency of one or more wireless sensors may be measured, and relative humidity of the soil may be determined based on the resonant frequency. Other embodiments are described and claimed.

### CROSS-REFERENCE TO RELATED APPLICATION

This application claims priority to U.S. Provisional Application Ser. No. 63/454,369, filed Mar. 24, 2023, the entire disclosure of which is hereby incorporated by reference.

### BACKGROUND

Agricultural production is the largest consumer of fresh water worldwide. Precision agriculture techniques include using soil water content sensors for monitoring water content at various depths in crop farmland. Current techniques for soil water content sensing include time-domain reflectometry (TDR) and frequency domain reflectometry (FDR), which typically require relatively expensive dataloggers used with multiple probes throughout the farm field. Those probes may be connected with lengthy wires, or for wireless applications may include individual batteries. Other soil water content sensing technologies include tensiometers that measure soil water potential, and solid state sensors that measure water diffusion, which are both commonly based on electrical resistance sensors and similarly require lengthy wires or batteries for each sensor. Similarly, typical humidity sensors used in agriculture are resistance and capacitance sensors, and also require long wires to connect the probe to an external device or the use of batteries as a power module of the sensor. Remote water content sensing technologies such as ground penetrating radar (GPR) typically have low spatial resolution, and may not be capable of measuring water content below the surface layer of the soil.

### SUMMARY

According to one aspect a device for wireless soil humidity sensing includes a flat surface capacitor and an inductor coupled to the flat surface capacitor. The flat surface capacitor comprises a dielectric material having a high relative dielectric permittivity, wherein the relative dielectric permittivity of the dielectric material changes in response to changes in environmental humidity.

In some embodiments, the dielectric material has a relative dielectric permittivity of above 1000. In some embodiments, the dielectric material comprises a ceramic material. In some embodiments, the dielectric material comprises a core-shell ceramic comprising BaTiO₃ and SiO₂.

In some embodiments, the dielectric material of the flat surface capacitor comprises a first side and a second side opposite the first side, and the flat surface capacitor further comprises a first electrode and a second electrode positioned on the first side of the dielectric material and separated by a first distance. In some embodiments, the flat surface capacitor further comprises an encapsulation layer that covers the first side of the dielectric, the first electrode, the second electrode, and the inductor, and wherein the second side of the dielectric is exposed to the environment. In some embodiments, the encapsulation layer comprises an epoxy material.

According to another aspect, a method for wireless soil humidity sensing includes distributing a wireless sensor device in a soil environment, wherein the wireless sensor device comprises a flat surface capacitor comprising a dielectric material having a high relative dielectric permittivity, wherein the relative dielectric permittivity of the dielectric material changes in response to changes in environmental humidity, and an inductor coupled to the flat surface capacitor; interrogating the wireless sensor device with a varying electromagnetic field; determining a resonant frequency of the wireless sensor device in response to interrogating the wireless sensor device; and determining a relative humidity of the soil environment as a function of the resonant frequency.

In some embodiments, the dielectric material of the wireless sensor device has a relative dielectric permittivity of above 1000. In some embodiments, the dielectric material of the wireless sensor device comprises a core-shell ceramic comprising BaTiO₃ and SiO₂.

In some embodiments, interrogating the wireless sensor device comprises coupling the inductor of the wireless sensor device with a pickup coil of an interrogator device; and determining the resonant frequency comprises determining a frequency having a lowest impedance of the coupled inductor and pickup coil. In some embodiments, determining the relative humidity of the soil environment as a function of the resonant frequency comprises determining a capacitance of the wireless sensor device as a function of the resonant frequency; and determining the relative humidity as a function of the capacitance.

In some embodiments, the method further comprises distributing a plurality of wireless sensor devices in the soil environment at different locations and depths; interrogating the plurality of wireless sensor devices with the varying electromagnetic field; determining one or more resonant frequencies of the plurality of wireless sensor devices in response to interrogating the plurality of wireless sensor devices; and determining one or more relative humidity values of the soil environment as a function of the one or more resonant frequencies.

According to another aspect, a method for manufacturing a wireless soil humidity sensor includes coating barium titanate (BaTiO₃) nanopowder particles with a layer of silicon dioxide (SiO₂) to create core-shell nanopowders; sintering the core-shell nanopowders to create a sintered tablet; cutting and polishing the sintered table to create a ceramic specimen having a first side and second side opposite the first side; depositing a first electrode and a second electrode on the first side of the ceramic specimen, wherein the first electrode and the second electrode comprise gold (Au); and coupling an inductor to the first electrode and the second electrode.

In some embodiments, the method further comprises encapsulating the first side of the ceramic specimen, the first electrode, the second electrode, and the inductor with an epoxy layer.

In some embodiments, the BaTiO₃ nanopowder particles have a diameter of about 140 nm. In some embodiments, the sintered tablet has a diameter of about 20 mm and a thickness of about 5 mm, wherein the ceramic specimen has a surface area of about 18 mm² and a thickness of about 1100 μm.

In some embodiments, sintering the core-shell nanoparticles comprises sintering the core-shell nano-particles at 1050° C. at 50 MPa for about 5 minutes. In some embodiments, coating the BaTiO₃ nanopowder particles with the layer of SiO₂ comprises coating by atomic layer deposition. In some embodiments, depositing the first electrode and the second electrode comprises sputtering.

### BRIEF DESCRIPTION OF THE DRAWINGS

The concepts described in the present disclosure are illustrated by way of example and not by way of limitation in the accompanying figures. For simplicity and clarity of illustration, elements illustrated in the figures are not necessarily drawn to scale. For example, the dimensions of some elements may be exaggerated relative to other elements for clarity. Further, where considered appropriate, reference labels have been repeated among the figures to indicate corresponding or analogous elements. The detailed description particularly refers to the accompanying figures in which:

FIG. 1 is a simplified schematic diagram of a system for wireless soil water content sensing;

FIG. 2 is a perspective view of a flat surface capacitor of the system of FIG. 1;

FIG. 3 is a cross-sectional view of the flat surface capacitor of FIG. 2;

FIG. 4 is a circuit diagram of the system of FIG. 1;

FIG. 5 is a cross-sectional view of another embodiment of a wireless sensor of the system of FIG. 1;

FIG. 6 is a simplified flow diagram of one embodiment of a method for wireless soil water content sensing that may be performed using the system of FIG. 1;

FIG. 7 is a simplified flow diagram of one embodiment of a method for manufacturing a sensor of the system of FIG. 1;

FIG. 8 is a plot illustrating experimental results that may be achieved with sensor of the system of FIG. 1; and

FIG. 9 is a plot illustrating additional experimental results that may be achieved with sensor of the system of FIG. 1.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the present disclosure.

References in the specification to “one embodiment,” “an embodiment,” “an example embodiment,” etcetera, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

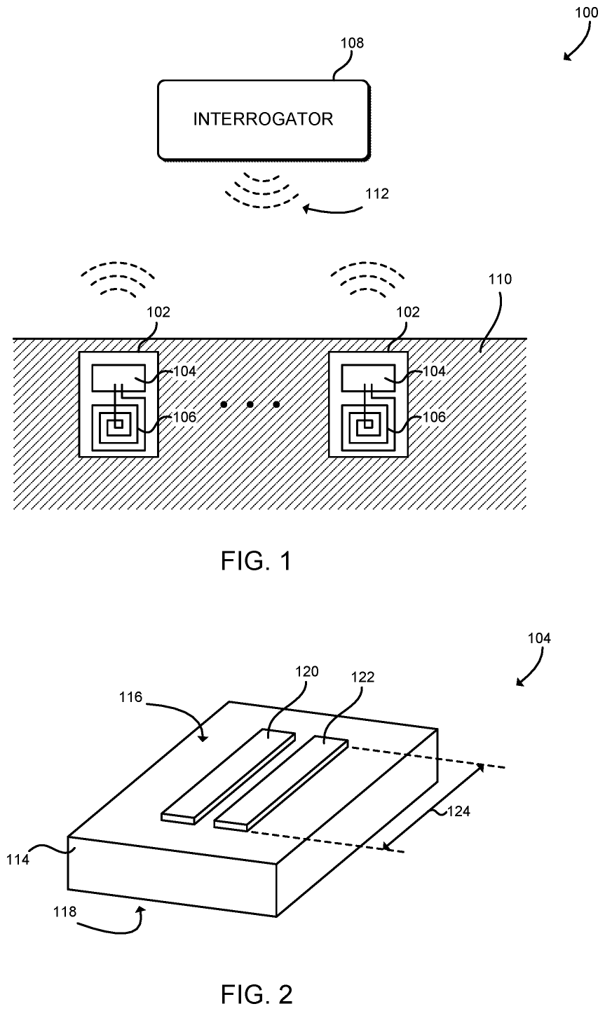

Referring now to FIG. 1, a simplified schematic diagram of a system 100 for wireless soil water content sensing is shown. The system 100 includes multiple wireless sensors 102. Each of the sensors includes a capacitor 104 coupled to an inductor 106. The system 100 further includes a wireless interrogator 108 that may be used to read out water content information from the sensors 102. In use, as shown, the sensors 102 may distributed over a geographical area underneath soil 110. For example, the sensors 102 may be buried throughout a farm field or other agricultural area. As described further below, each of the sensors 102 is an inductor-capacitor (LC) resonant circuit with a resonant frequency that changes with humidity of the environment, which in the illustrative embodiment is the soil 110. The interrogator 108 generates a varying magnetic field 112 that interacts with the sensors 102 and detects the resonant frequency of one or more of the sensors 102. Based on the resonant frequency, the interrogator 108 and/or other device determines detected humidity, and thus soil water content. Accordingly, the system 100 provides real-time measurement of soil water content using inexpensive, passive sensors. By using inexpensive sensors, the system 100 may provide accurate average measurements over a large area, or may be used to provide detailed measurement data with high spatial resolution, including at multiple depths. Additionally, the sensors of the system 100 provide wireless sensing without requiring individual batteries in the sensors, which may reduce chemical pollution in soil as compared to other water content sensors with batteries.

As shown in FIG. 1, each sensor 102 includes a capacitor 104. Each capacitor 104 includes a dielectric material having a high dielectric relative permittivity as compared to the surrounding environment, which may include air and soil. For example, in an illustrative embodiment, the dielectric material may have a relative dielectric permittivity of about 1,000, above 1,000, or above 10,000. Relative dielectric permittivity is a measure of the absolute permittivity of the material compared to permittivity of vacuum. Accordingly, air has a relative dielectric permittivity of about 1.0, and many types of soil have a relative permittivity of about 3-80, depending on the type of soil and the soil water content. Accordingly, the dielectric material of the capacitor 104 has a dielectric permittivity that is much larger than that of air or soil. Because the dielectric material has a relative dielectric permittivity much larger than the surrounding environment (i.e., air or soil), the capacitance of the capacitor 104 does not appreciably change when the capacitor 104 is covered in soil, including soil with varying water content.

Additionally, the dielectric permittivity of the dielectric material of the capacitor 104 changes in response to changes in humidity in the environment. This change in permittivity changes the capacitance of the capacitor 104. The capacitor 104 may be formed using techniques to increase the surface area of the dielectric material that interacts with the environment, in order to reduce the time needed to sense changes in humidity. For example, in the illustrative embodiment, the dielectric material may be a porous ceramic material. Of course, in other embodiments, the capacitor 104 may be formed with any other appropriate dielectric material having a high dielectric permittivity that changes in response to environmental humidity.

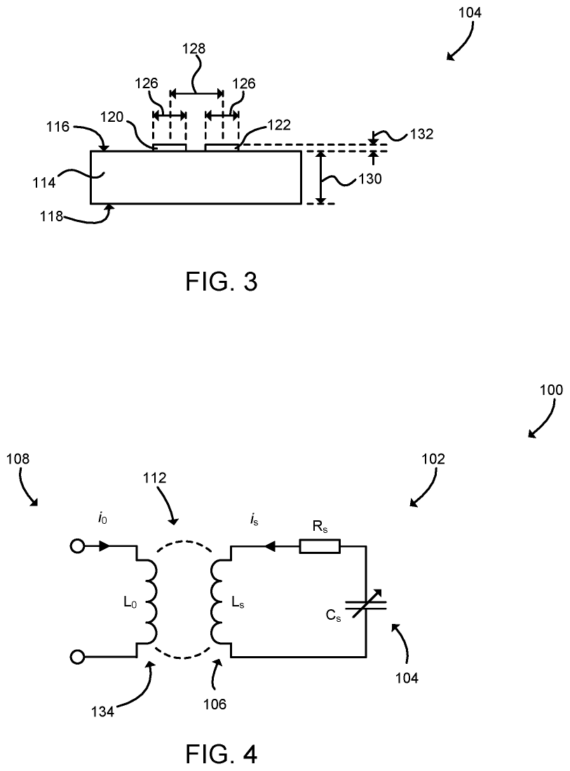

Referring now to FIGS. 2 and 3, one potential embodiment of a flat surface capacitor 104 that may be used with the system 100 is shown. The illustrative capacitor 104 includes a dielectric material 114 that is formed as a flat specimen having two opposing sides 116, 118. Two electrodes 120, 122 are positioned on top of the surface 116. The surface 118 is exposed to the environment surrounding the capacitor 104. As best shown in FIG. 2, each electrode 120, 122 has a length 124, and as best shown in FIG. 3, each electrode has a width 126. The electrodes 120, 122 are separated by a constant distance 128 between the midpoint of each electrode 120, 122. The dielectric material 114 has a thickness 130, and the electrodes 120, 122 have a thickness 132. Capacitance C for the flat surface capacitor 104 may be determined using Equation 1, below. In Equation 1, l is the length 124, ε_{r(eff)} is the effective relative dielectric permittivity of the dielectric material 114, 80 is the dielectric permittivity of vacuum, d is the distance 128, w is the width 126, and t is the thickness 132.

$$\frac{c}{l} \approx \frac{π {ε}_{r ( e f f )} {ε}_{0}}{ln ( \frac{π ( d - w )}{w + t} + 1 )} \qquad ( 1 )$$

In the illustrative embodiment, the dielectric material 114 is a barium titanate (BaTiO₃ or BTO)-silicon dioxide (SiO₂) ceramic. More particularly, the dielectric material 114 is a BTO-SiO₂ core-shell nanocomposite ceramic. The electrodes 120, 122 are illustratively formed from gold. In other embodiments, the electrodes 120, 122 may be formed from any other electrically conductive material. One potential embodiment of a method for manufacturing the capacitor 104 shown in FIGS. 2-3 is described further below in connection with FIG. 7.

As described above, the surface 118 of the capacitor 104 is not covered by the electrodes 120, 122 and is thus exposed to the environment surrounding the capacitor 104. Accordingly, air and water molecules from the surrounding environment may enter the dielectric material 114 through the exposed side 118. As compared to parallel plate capacitors or other capacitor arrangements in which the dielectric material is covered by the electrodes or other components, the flat surface capacitor 104 may react more quickly to changes in humidity. Additionally or alternatively, reducing the thickness 130 of the dielectric material 114 may allow the capacitor 104 to react more quickly to changes in relative humidity. For example, in many embodiments, much of the capacitance of the flat surface capacitor 104 is due to the permittivity of a relatively small portion of the dielectric material 114 between the electrodes 120, 122 near the surface 116. Reducing the thickness 130 may reduce the time needed for water molecules from the external environment to reach this portion of the dielectric material 114 from the opposing side 118.

Referring again to FIG. 1, as discussed above, the sensor 102 includes an inductor 106. The inductor may be embodied as one or more coils, loops, or other inductive electronic devices that may be coupled to the capacitor 104 to form an inductor-capacitor resonant circuit. For example, the inductor 106 may be a spiral inductor formed from conductive material which may be deposited in some embodiments on printed circuit board material. The inductance of the inductor 106 may be tuned such that the resonant frequency of the sensor 102 is within a predetermined range, which may be selected for efficiency, sensitivity, or for other factors. Illustratively, the inductance of the inductor 106 may be tuned such that the resonant frequency for the sensor 102 when the capacitor 104 is in a dry environment (e.g., below 15% relative humidity) is at a relatively low frequency, such as about 100 Hz, 500 Hz, 1 kHz, below 10 kHz, or another relatively low value. As described further below, at these frequencies, the permittivity of the dielectric material 114 may exhibit relatively large changes in response to changes in relative humidity, for example changing by at least an order of magnitude from dry conditions to 85% relative humidity. Additionally, lower resonant frequencies may exhibit lower loss and thus higher energy efficiency. Accordingly, such systems may have relatively high sensitivity with low loss.

The system 100 further includes an interrogator 108. The interrogator 108 may be embodied as any type of device capable of performing the operations described herein. Accordingly, the interrogator 108 may include or otherwise be embodied as a microcontroller, microprocessor, digital signal processor, or other computing device. The interrogator 108 may further include one or more signal generators, analog-to-digital converters, radio interfaces, and/or other electronic components commonly associated with generating and receiving electromagnetic signals. In particular, the interrogator 108 may include one or more sensor coils, readout coils, or other components capable of interacting with the wireless sensors 102. For example, the interrogator 108 may include one or more coils or other inductors capable of generating a varying magnetic field, magnetically coupling with the inductor 106 of one or more sensors 102, and measuring the response of the sensor 102. The interrogator 108 may include one or more drive coils capable of generating the varying magnetic field and one or more readout coils capable of magnetically coupling with the sensors 102. In some embodiments, those functions may be performed by a single readout coil. The interrogator 108 may detect the resonant frequency of the sensor 102, for example, by monitoring the impedance or input return loss of the readout coil.

Referring now to FIG. 4, a circuit diagram illustrating interactions between the interrogator 108 and a sensor 102 of the system 100 is shown. As shown, the sensor 102 is modeled as an R-L-C circuit including resistor Rₛ, inductor Lₛ, which is illustratively the inductor 106, and variable capacitor Cₛ, which is illustratively the flat surface capacitor 104. Accordingly, the sensor 102 has a resonant frequency fᵣ that depends on the capacitance of the capacitor 104, as shown in Equation 2, below. The diagram illustrates inductive coupling between the interrogator 108 and a sensor 102.

$${f}_{r} = \frac{1}{2 π \sqrt{{L}_{s} {C}_{s}}} \qquad ( 2 )$$

As shown, the interrogator 108 includes an inductor L₀, which corresponds to a readout coil 134 of the interrogator 108. When the interrogator 108 outputs a varying magnetic field 112, the readout coil 134 couples with the inductor 106 of the sensor 102. As shown, this coupling includes a current i₀ that flows through the readout coil 134 and an induced current is that flows through the inductor 106. There may be a coupling constant associated with the coils 134, 106 that does not change with frequency. However, overall impedance of the sensor 102 changes with frequency. Thus, for a signal from the interrogator 108 with the same intensity but a different frequency, the current in the coils 106, 134 depends on the frequency. Accordingly, the coupling between the coils 106, 134 results in a peak (or a lowest loss) in the current at the resonant frequency fᵣ of the sensor 102. Accordingly, the interrogator 108 may detect the resonant frequency fᵣ of the sensor 102. As described above, this resonant frequency fᵣ changes based on capacitance of the capacitor 104 and thus based on relative humidity of the environment of the sensor 102. Relative humidity of the soil 110 or other environment surrounding the sensor 102 may be determined based on the measured resonant frequency fᵣ, for example by the interrogator 108 or by another computing device.

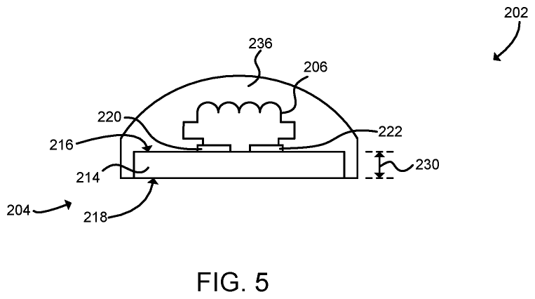

Referring now to FIG. 5, another embodiment of a wireless sensor 202 is shown. The wireless sensor 202 may be used in the system 100 in addition to or as an alternative to one or more of the sensors 102. Accordingly, the sensor 202 includes many of the same components of the wireless sensor 102, including a capacitor 204 and an inductor 206. The features of those components are similar to the components of the wireless sensor 102, and are illustrated with similar reference numbers in FIG. 5. The descriptions of the components of the wireless sensor 102 above are equally applicable to the wireless sensor 202 and are thus not repeated herein in order not to obscure the disclosure.

Additionally, the wireless sensor 202 includes an encapsulation layer 236 that covers the side 116 of the capacitor 104, including the electrodes 220, 222 positioned on the side 116, as well as the capacitor 206. The encapsulation layer 236 is illustratively epoxy, but may be embodied as any nonconductive, environmentally resistant material. The encapsulation layer 236 provides protection from the environment to various components of the wireless sensor 202. Additionally, the encapsulation layer 236 may provide mechanical support to the wireless sensor 202. Particularly in embodiments in which the dielectric material 214 of the capacitor 204 is a relatively brittle ceramic or ceramic composite, the encapsulation layer 236 may prevent breakage of the dielectric material 214.

As shown, the encapsulation layer 236 does not cover the side 218 of the dielectric material 214, and thus the side 218 remains exposed to the environment. Accordingly, the wireless sensor 202 may exhibit similar performance, speed, and sensitivity for humidity sensing as compared to the wireless sensor 102 of FIGS. 2-3. Further, in some embodiments, the thickness 230 of the dielectric material 214 may be reduced compared to the thickness 130 of the dielectric material 114 in the sensor 102, because the encapsulation layer 236 provides mechanical support. As described above, by reducing the thickness 230, the wireless sensor 202 may increase the speed at which it senses changes in humidity in the environment.

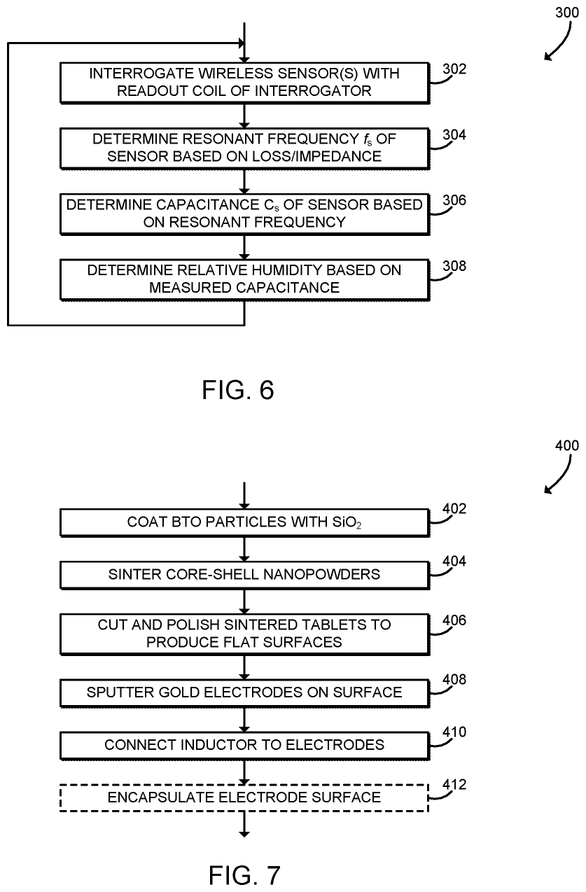

Referring now to FIG. 6, one illustrative embodiment of a method 300 for soil water content sensing with the system 100 is shown as a simplified flow diagram. The method 300 is illustrated as a series of blocks 302-308, some of which may be optionally performed in some embodiments. It will be appreciated by those of skill in the art that some embodiments of the method 300 may include additional or different processes and sub-processes.

The method 300 begins with block 302, in which the interrogator 108 interrogates one or more wireless sensors 102, 202 with a readout coil 134 of the interrogator 108. The sensors 102, 202 may be distributed within soil 110 throughout an area such as an agricultural farm or other area. The interrogator 108 may generate a varying magnetic field at various frequencies and measure the response of one or more sensors 102, 202. In particular, the interrogator 108 may determine loss or otherwise measure impedance of the RLC circuit for each of the one or more sensors 102, 202.

In block 304, the interrogator 108 or other computing device determines the resonant frequency fᵣ of one or more sensors 102, 202 based on the measured loss or impedance. The resonant frequency fᵣ may be a frequency at which one or more of the sensors 102, 202 exhibit the lowest loss. The resonant frequency fᵣ may be determined as the peak point of a phase spectrum picked up by the readout coil. The interrogator 108 or other computing device may determine an average resonant frequency fᵣ for multiple sensors 102, 202, which may provide a more accurate measurement of water content for an area. Additionally or alternatively, the interrogator 108 or other computing device may determine multiple resonant frequency fᵣ values based on sensors 102, 202 from different locations, which may be used for water content mapping or other water content analysis.

In block 306, the interrogator 108 or other computing device determines capacitance Cₛ of the capacitor 104, 204 as described above. For example, the interrogator 108 or other computing device may determine the capacitance Cₛ using Equation 2, above. In block 308, the interrogator 108 or other computing device determines relative humidity based on the measured capacitance Cₛ of the capacitor 104, 204. As described above, dielectric permittivity of the dielectric material 114, 214 changes in response to changes in the relative humidity of the environment surrounding the sensor 102, 202. This change in permittivity changes the capacitance of the capacitor 104, 204. Because, as described above, the permittivity of the dielectric material 114, 214 is much larger than the surrounding soil 110, this change in permittivity (and thus capacitance) is not practically affected by the presence of soil 110 surrounding the sensor 102, 202.

The interrogator 108 or other computing device may use any appropriate technique to calculate the relative humidity. For example, the interrogator 108 or other computing device may include a table of predetermined values for converting capacitance to relatively humidity, or may be otherwise calibrated based on measured performance of the sensors 102, 202. Additionally or alternatively, although illustrated as determining capacitance, it should be understood that in some embodiments the interrogator 108 or other computing device may determine relative humidity based on a different measured quantity. For example, in some embodiments, the interrogator 108 or other computing device may determine relative humidity directly based on measured resonant frequency fᵣ. After determining relative humidity, the method 300 loops back to block 302 to continue wirelessly sensing water content.

Referring now to FIG. 7, one illustrative embodiment of a method 400 for manufacturing a sensor 102, 202 is shown as a simplified flow diagram. The method 400 is illustrated as a series of blocks 402-410, some of which may be optionally performed in some embodiments (and, thus, are shown in dashed lines). It will be appreciated by those of skill in the art that some embodiments of the method 400 may include additional or different processes and sub-processes.

The method 400 begins with block 402, in which barium titanate (BTO) particles are coated with silicon dioxide (SiO₂). The BTO particles are illustrative BTO nano particles having a diameter of 140 nm. The BTO particles are coated with SiO₂ using atomic layer deposition.

In block 404, the core-shell BTO-SiO₂ particles are sintered to form a ceramic tablet. Illustratively, the BTO-SiO₂ particles are sintered at 1050° C. for five minutes at 50 MPa using a direct current sintering furnace. Samples may be heated at a rate of 100° C./min to 950° C. and held for two minutes and then heated at a rate of 50° C./min to 1050° C. to avoid overshooting the final temperature. Illustratively, the sintered tablet may have a diameter of about 20 mm and a thickness of 5 mm. Other sizes or shapes are possible.

In block 406, the sintered ceramic composite material is cut and polished to form a ceramic specimen with two flat, opposing surfaces. Illustratively, the material may be diced or otherwise cut with a wafering blade into rectangular specimens having a surface area of about 18 mm² a thickness of about 900-1100 μm. In block 408, two gold electrodes are sputtered onto one of the surfaces of the ceramic specimen. Illustratively, each electrode has a rectangular shape having length and width of three by one millimeter. Illustratively, the electrodes are separated by a uniform gap of 150 μm. After forming the electrodes on the ceramic specimen, a capacitor 104, 204 has been formed. In block 410, an inductor 106, 206 is coupled to the electrodes on the surface of the capacitor 104, 204. The inductor may be soldered or otherwise connected to the electrodes. After connecting the inductor 106, 206 to the capacitor 104, 204, a wireless sensor 102, 202 has been formed, and in some embodiments the method 400 may be completed.

In some embodiments, in block 412, the surface of the capacitor 104, 204 having the electrodes is encapsulated with an epoxy encapsulation layer. The epoxy encapsulation layer may also encapsulate the inductor 106, 206 or other components of the sensor 202. The side of the ceramic specimen opposite the electrodes is not covered by epoxy and thus remains exposed to the environment. After encapsulating the surface of the capacitor, a wireless sensor 202 has been formed, and the method 400 is completed.

After manufacturing a wireless sensor 102, 202 (or a capacitor 104, 204), in some embodiments the sensor 102, 202 (or capacitor 104, 204) may be subjected to a pretreatment process including multiple humidity cycles, in which the environment surrounding the sensor 102, 202 (or capacitor 104, 204) is brought from a dry condition (e.g., less than 8% relative humidity) to a high humidity condition (e.g., 85% relative humidity) and back to the dry condition. These humidity cycles may eliminate humidity sensing hysteresis and ensure that the sensor 102, 202 (or capacitor 104, 204) produces stable responses to humidity. For example, in some embodiments, the wireless sensor 102, 202 may be subjected to 10 humidity cycles over 20 days, after which the sensor 102, 202 may be used as described above. Continuing that example, after pretreatment the sensor 102, 202 may exhibit stable humidity responses for an extended number of humidity cycles. In an experiment, an illustrative sensor 102, 202 exhibited stable humidity sensing for at least 23 additional cycles over 46 days after the 10-cycle pretreatment process.

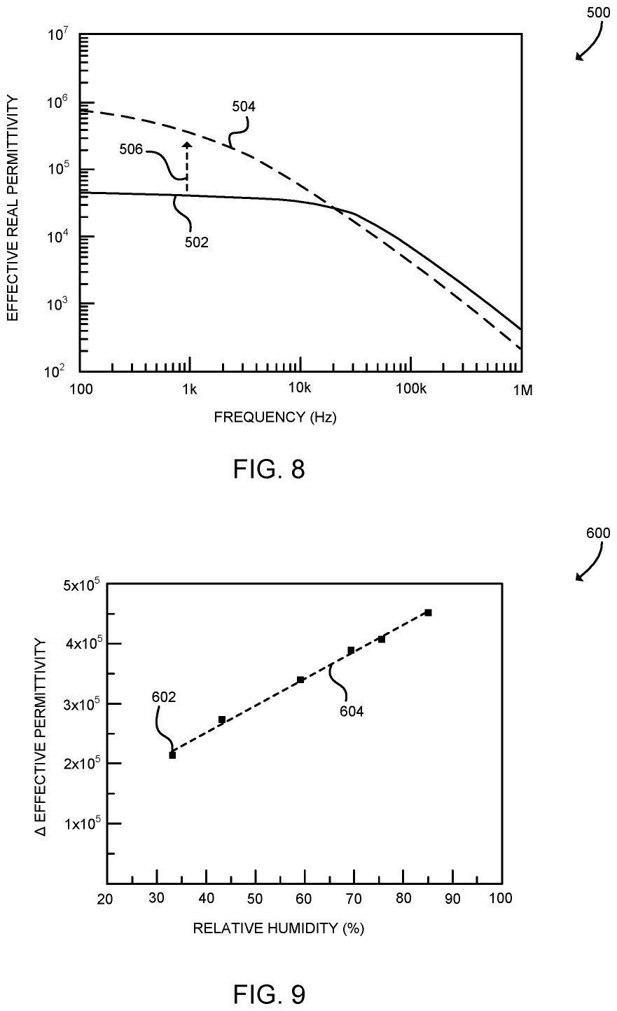

Referring now to FIG. 8, plot 500 illustrates experimental results that may be achieved by the system 100. In an experiment, a wireless sensor 102, 202 including a flat surface capacitor 104, 204 was measured in an environment with humidity in the range between a dry condition with less than 8% relative humidity and a humid condition with 85% relative humidity. In the experiment, capacitance of the sensor 102, 202 was measured with a precision impedance analyzer over a frequency range from 100 Hz to 1 MHz. Using the measured capacitance value, effective real permittivity of the capacitor 104, 204 was determined using Equation 1, above. Curve 502 illustrates the measured effective real permittivity in the dry condition, and curve 504 illustrates the measured effective real permittivity in the 85% relative humidity condition. As shown, in either of the dry condition or the 85% relative humidity condition, permittivity gradually decreases at low frequency and then dramatically decreases at high frequency (e.g., above about 20 kHz). When comparing the dry condition to the 85% relative humidity condition, permittivity in the 85% relative humidity condition is greater than permittivity for the dry condition for frequencies lower than about 20 kHz. For frequencies above about 20 kHz, permittivity in the 85% relative humidity condition is lower than permittivity for the dry condition. As shown, at 100 Hz, permittivity for the 85% relative humidity is more than an order of magnitude greater than permittivity for the dry condition. At 1 KHz, permittivity for the 85% relative humidity is still about an order of magnitude greater than permittivity for the dry condition. Note that at a particular frequency (e.g., about 20 kHz), permittivity does not change between the dry condition and the 85% relative humidity condition.

When the humidity of the environment surrounding the sensor 102, 202 changes from dry to 85% relative humidity, the permittivity curve changes from the curve 502 to the curve 504, as represented by arrow 506. When used with a flat-surface capacitor 104, 204 as described above, this change occurs quickly, for example within several minutes (e.g., less than 10 minutes, less than 5 minutes, less than 3 minutes, or less than 1 minute). For example, in the illustrative experiment, the curve 502 changed to very nearly reach the curve 504 within about three minutes. Similarly, when the environment surrounding the sensor 102, 202 changes from 85% relative humidity to a dry condition, the permittivity curve changes from the curve 504 to the curve 502. This change also occurs relatively quickly for the flat-surface capacitor 104, 204. For example, in the illustrative experiment, the curve 504 changed to very nearly reach the curve 502 in about 5-7 minutes.

Note that for typical ceramic humidity sensors, it is believed that total capacitance is largely influenced by grain boundary potential barrier capacitance, which varies as the amount of water molecules bonded in the grain boundaries varies with relative humidity. However, this mechanism is believed to influence dielectric permittivity of the sensor over the entire frequency spectrum. Accordingly, it is unexpected that the permittivity of the sensor 102, 202 increases with increased relative humidity for lower frequencies but decreases with increased relative humidity for higher frequencies.

In another experiment (not shown), similar tests of sensors 102, 202 were performed with the sensors 102, 202 exposed to various media. In particular, effective permittivity over the frequency range of 100 Hz to 1 MHz was measured as described above for sensors 102, 202 that were freestanding in air, covered by sand, and covered by topsoil, in both the dry condition and in the 85% relative humidity condition. In this experiment, it was determined that there were no measured differences in effective permittivity of the sensors 102, 202 based on being exposed in air or covered in soil (sand or topsoil).

Referring now to FIG. 9, plot 600 illustrates additional experimental results that may be achieved by the system 100. The plot 600 illustrates the measured change in effective real permittivity for values of relative humidity as compared to the dry condition, measured at a frequency of 500 Hz. Data points 602 represent measured changes in effective permittivity for particular relative humidity percentages using frequency 500 Hz, and line 604 represents a linear best-fit line for the data points 602. As shown, the plot 600 demonstrates a linear relation between relative humidity and the change in effective permittivity. Similar plots 600 may be created for other frequencies, such as 100 Hz, 1 kHz, 3 kHz, 100 kHz, or a different frequency. In an experiment, it was found that the lower frequencies of 100 Hz, 500 Hz, and 1 kHz had a better linear fitting (e.g., higher coefficient of determination R²) as compared to higher frequencies. Additionally, the sensor 102, 202 exhibited lower dielectric loss at those lower frequencies. Accordingly, the low-frequency range from 500 Hz to 1 kHz may provide better overall humidity sensing performance with high sensitivity and low loss.

While certain illustrative embodiments have been described in detail in the figures and the foregoing description, such an illustration and description is to be considered as exemplary and not restrictive in character, it being understood that only illustrative embodiments have been shown and described and that all changes and modifications that come within the spirit of the disclosure are desired to be protected. There are a plurality of advantages of the present disclosure arising from the various features of the apparatus, systems, and methods described herein. It will be noted that alternative embodiments of the apparatus, systems, and methods of the present disclosure may not include all of the features described yet still benefit from at least some of the advantages of such features. Those of ordinary skill in the art may readily devise their own implementations of the apparatus, systems, and methods that incorporate one or more of the features of the present disclosure.

1. A device for wireless soil humidity sensing, the device comprising:
a flat surface capacitor comprising a dielectric material having a high relative dielectric permittivity, wherein the relative dielectric permittivity of the dielectric material changes in response to changes in environmental humidity; and
an inductor coupled to the flat surface capacitor.

2. The device of claim 1, wherein the dielectric material has a relative dielectric permittivity of above 1000.

3. The device of claim 2, wherein the dielectric material comprises a ceramic material.

4. The device of claim 3, wherein the dielectric material comprises a core-shell ceramic comprising BaTiO₃ and SiO₂.

5. The device of claim 1, wherein:
the dielectric material of the flat surface capacitor comprises a first side and a second side opposite the first side; and
the flat surface capacitor further comprises a first electrode and a second electrode positioned on the first side of the dielectric material and separated by a first distance.

6. The device of claim 5, wherein the flat surface capacitor further comprises an encapsulation layer that covers the first side of the dielectric, the first electrode, the second electrode, and the inductor, and wherein the second side of the dielectric is exposed to the environment.

7. The device of claim 6, wherein the encapsulation layer comprises an epoxy material.

8. A method for wireless soil humidity sensing, the method comprising:
distributing a wireless sensor device in a soil environment, wherein the wireless sensor device comprises (i) a flat surface capacitor comprising a dielectric material having a high relative dielectric permittivity, wherein the relative dielectric permittivity of the dielectric material changes in response to changes in environmental humidity, and (ii) an inductor coupled to the flat surface capacitor;
interrogating the wireless sensor device with a varying electromagnetic field;
determining a resonant frequency of the wireless sensor device in response to interrogating the wireless sensor device; and
determining a relative humidity of the soil environment as a function of the resonant frequency.

9. The method of claim 8, wherein the dielectric material of the wireless sensor device has a relative dielectric permittivity of above 1000.

10. The method of claim 9, wherein the dielectric material of the wireless sensor device comprises a core-shell ceramic comprising BaTiO₃ and SiO₂.

11. The method of claim 8, wherein:
interrogating the wireless sensor device comprises coupling the inductor of the wireless sensor device with a pickup coil of an interrogator device; and
determining the resonant frequency comprises determining a frequency having a lowest impedance of the coupled inductor and pickup coil.

12. The method of claim 8, wherein determining the relative humidity of the soil environment as a function of the resonant frequency comprises:
determining a capacitance of the wireless sensor device as a function of the resonant frequency; and
determining the relative humidity as a function of the capacitance.

13. The method of claim 8, further comprising:
distributing a plurality of wireless sensor devices in the soil environment at different locations and depths;
interrogating the plurality of wireless sensor devices with the varying electromagnetic field;
determining one or more resonant frequencies of the plurality of wireless sensor devices in response to interrogating the plurality of wireless sensor devices; and
determining one or more relative humidity values of the soil environment as a function of the one or more resonant frequencies.

14. A method for manufacturing a wireless soil humidity sensor, the method comprising:
coating barium titanate (BaTiO₃) nanopowder particles with a layer of silicon dioxide (SiO₂) to create core-shell nanopowders;
sintering the core-shell nanopowders to create a sintered tablet;
cutting and polishing the sintered table to create a ceramic specimen having a first side and second side opposite the first side;
depositing a first electrode and a second electrode on the first side of the ceramic specimen, wherein the first electrode and the second electrode comprise gold (Au); and
coupling an inductor to the first electrode and the second electrode.

15. The method of claim 14, further comprising encapsulating the first side of the ceramic specimen, the first electrode, the second electrode, and the inductor with an epoxy layer.

16. The method of claim 14, wherein the BaTiO₃ nanopowder particles have a diameter of about 140 nm.

17. The method of claim 14, wherein the sintered tablet has a diameter of about 20 mm and a thickness of about 5 mm, wherein the ceramic specimen has a surface area of about 18 mm² and a thickness of about 1100 μm.

18. The method of claim 14, wherein sintering the core-shell nanoparticles comprises sintering the core-shell nano-particles at 1050° C. at 50 MPa for about 5 minutes.

19. The method of claim 14, wherein coating the BaTiO₃ nanopowder particles with the layer of SiO₂ comprises coating by atomic layer deposition.

20. The method of claim 14, wherein depositing the first electrode and the second electrode comprises sputtering.
